(19) United States
(12) Patent Application Publication
(10) Pub. No.: US 2005/0112138 A1
(43) Pub. Date: May 26, 2005

(54) Aids vaccines

(76) Inventor: Nancy L. Haigwood, Seattle, WA (US)

Correspondence Address: Thomas F. Broderick, Esq.;Seattle Biomedical Research Institute, 307 Westlake Avenue North, Suite 500, Seattle, WA, 98109-5219, US

(21) Appl. No.: 10/719,004

(22) Filed: Nov. 21, 2003

(51) Int. Cl. A61K 39/21

(52) U.S. Cl. 424/188.1

(57) ABSTRACT

Vaccination by immunizing a mammalian host with a first HIV envelope immunogen having a minimum number of N- and O-linked glycosylation sites, followed by one or more booster immunizations with at least one second HIV envelope immunogen having more glycosylation sites than the first immunogen.

This invention was made with government support under grant P01 AI-26503 awarded by the Public Health Service, National Institutes of Health. The government has certain rights in the invention.

### FIELD OF THE INVENTION

The invention relates to HIV vaccines, and particularly to improved immunogens for generating HIV neutralizing antibodies (NtAbs).

### BACKGROUND OF THE INVENTION

The HIV Envelope glycoprotein is an oligomeric glycoprotein that is expressed from the viral env gene as a 160 kDa fusion protein or Envelope (Env) inserted into the HIV-infected cell membrane. This protein is processed into a gp120 surface protein (SU) and a gp41 transmembrane-bound transmembrane protein (TM). Once expressed on the surface of HIV-infected cells, the Env oligomer coats the HIV virion through budding of the viral core from within the cell at sites on the cell surface where Env is clustered. Virions then utilize the Env protein to infect new cells through binding to specific cellular receptors, the human CD4 receptor and a coreceptor from the chemokine family, most usually CCR5. Fusion of the viral Envelope with the target cell is accomplished by first binding of Env to cellular receptors, followed by penetration of the cell membrane via a fusion domain at the N-terminus of the TM protein.

The SU is non covalently associated with the TM. Expression of Env using recombinant DNA has led to a number of different versions of Env that have been tested as vaccine immunogens. Full-length Env gp160 can be obtained by extraction of cellular membranes. The SU protein Env gp120 can be made as a monomer by truncating the env gene at the natural cleavage site for SU/TM. Additional mutations can be made to covalently attach SU and TM. One such mutation involves alteration of the SU/TM cleavage site at basic residues to prevent cleavage. Another mutation can be made to truncate the TM protein Env gp41 at the C-terminal amino acid prior to the transmembrane sequence, so that the hydrophilic N-terminal fusion domain can be expressed along with the SU gp120; this construct is typically named Env gp140 for its approximate size in kDa on polyacrylamide gels. Env gp140 can be made with or without an intact cleavage site for the cellular protease to utilize.

Phylogenetic analysis shows that HIV is highly variable in sequence, comparing sequences from different individuals. Comparison of viral sequences from infected persons worldwide has led to the classification of HIV into subtypes based on sequence homology. In addition to differences among HIV-infected individuals (interpatient variability), the virus is also variable within an individual (intrapatient variability). Interpatient variants in Env, the least conserved viral protein, can differ by as much as 20% at the amino acid level. Intrapatient variants are more similar to each other than to sequences from any other patient, and the constellation of variants in an individual is termed the “quasispecies.” Differences in HIV sequence are most pronounced the env gene, and within the env gene, variability is clustered in the gp120 SU protein in regions termed “hypervariable regions”. Due to the constraints of the Env protein to perform its binding and fusion functions, certain regions of Env must by necessity be conserved in all viral variants. The binding site for CD4 and gp120 has been shown to be a discontinuous epitope, with contact points found in three more conserved regions that are not contiguous in the amino acid sequence, but rather are brought into proximity when gp120 is folded into its native shape. More than half of the molecular weight of Env is carbohydrate, consisting of various forms of sugar moieties bound to asparagine (“Asn,” or “N”) residues. The addition of N-linked or O-linked sugars occurs during expression of Env and targeting and delivery of the protein to the cell membrane. For simplicity's sake, these will be referred to as “N-linked” in this application. Detailed biochemical analyses of the carbohydrate residues have shown that they differ at specific sites of Env and can be high mannose, complex, etc. (see FIG. 1). Certain N-linked sites are conserved between all characterized isolates to date, and these carbohydrate residues may contribute to the overall function of Env by influencing the three-dimensional structure of the glycoprotein oligomer.

Role ofNeutralizing Antibodies in Vaccine development for conferring immunity from HIV infection. Neutralizing antibodies (NtAbs) are defined as those antibodies that can block HIV from productively infecting target cells in vitro. NtAbs are currently measured to arise within the first 3-6 months of infection, after the appearance of binding antibodies directed to HIV, or seroconversion (Cecilia et al. 1999). All known NtAbs are directed to Env. In the course of infection, NtAbs are directed first at highly charged regions of Env, typically hypervariable regions of the SU Env protein. For this reason, the ability of these early NtAbs to neutralize HIV is restricted to HIV isolates that match in the variable regions. From infection studies in the chimpanzee and the macaque, it has been demonstrated that polyclonal NtAbs and monoclonal Abs (mAbs) that neutralize HIV can each fully block infection, if they are present in the body at high levels at the time of infection.

The role of NtAbs in virus blocking prior to infection versus virus control once HIV infection is established may be different. Once HIV infection is established, NtAbs can be shown to change over time in an individual. Initially, NtAbs neutralize the homologous virus, but the virus “escapes” from neutralization, followed by the development of NtAbs that neutralize the escape variants (Burns and Desrosiers 1994; Wang et al. 2002). The cycle of escape and containment is then repeated for the duration of the infection. Importantly, some patients who live for years with HIV infection develop NtAbs that block not only their own HIV variants but also HIV from other patients infected with HIV from the same subtype or lade (intrasubtype neutralization) AND also HIV from other patients infected with HIV from different subtypes (intersubtype neutralization). The mechanism for the generation of these intrasubtype and intersubtype NtAbs is not known, but their existence is well documented in the scientific literature (Moog et al. 1997; Kimura et al. 2002). Because these patients typically have not been exposed to sequences from other subtypes or clades, it has been hypothesized that the NtAbs work via recognition of conserved structures that are shared among the different subtypes. Indeed, sera from HIV-infected patients were shown to contain conformation-dependent NtAbs that can recognize sequentially diverse HIV subtypes (Steimer et al. 1991). The Env has been shown to change conformation in the process of binding to the receptor CD4, exposing neutralization determinants (Sullivan et al. 1998).

In SIV-infected macaques, the extent of viral Env evolution and the titer of NtAb correlate with the extent of persistent viremia (Hirsch et al. 1998). In HIV infection, the development of novel NtAbs during antiviral drug suppression requires the production of at least measurable levels of viral antigen (Kimura et al. 2002). HIV-infected patients vary in the levels of their intra- and intersubtype-specific NtAbs, and the extent of Env variation is also variable. As the virus evolves during the course of an HIV infection, the Env sequence changes and frequently alters both alters the number and the location of N-linked glycosylation sites. One of the key mutations in the HIV Env protein that is associated with neutralization escape is the addition and deletion of carbohydrate residues and/or charged residues in or near the V1 and V2 hypervariable regions and in the upstream C3 region (Wang et al. 2002). It has been shown that glycosylation mutants of SIV arising later in infection and that add carbohydrate can be more resistant to neutralization (Chackerian et al. 1997) and more pathogenic in vivo (Kimata et al. 1999). Hypervariable regions are involved in binding to coreceptors, and changes to these regions have been shown to influence the conformation of Env affecting subunit association, syncytium formation, and recognition by a neutralizing antibody (Sullivan et al. 1993; Wyatt et al. 1995).

Prior HIV vaccines have employed Env immunogens with limited clinical success. Soluble Envelopes (gp120) act as excellent immunogens to generate anti-Env antibodies, but typically generate only low levels of homologous NtAbs, sometimes intrasubtype NtAbs, but only very rarely intersubtype NtAbs (Haigwood et al. 1992; Mascola et al. 1996). To drive the immune response toward conserved regions, it was logical to immunize with an Env devoid of variable sequences, and such experiments were attempted with recombinant Env subunit proteins. It was demonstrated that at least one variable region was necessary to elicit detectable NtAbs in experimental animals (Haigwood et al. 1990). Failure to raise NtAbs to conserved regions was probably due to the denature immunogen, which did not preserve the shape of the remaining conserved, nonvariable regions. Approaches to overcoming the poor immunogenicity of conserved conformational determinants have included the use of primary virus Env gp120, and soluble oligomeric Envs (Stamatos et al. 1998), stabilized by disulfide bonds (Binley et al. 2000) or shortened to make Env gp140 by removing the transmembrane domain of the TM protein gp41 (Stamatatos et al. 2000). None of these approaches has resulted in immunogens that elicit broad immunity at the level seen in HIV-infected patients with high-level intersubtype NtAbs. Another approach to altering the repertoire of NtAbs was to draw immunity away from the immunogenic variable region V3 by masking the region with carbohydrate (Garrity et al. 1997). The masking succeeded in driving immunity to other variable regions that were more immunogenic than in the wild type Env, but no cross-neutralizing responses were obtained.

The Envs from late stage patients who have antibodies that can neutralize intra- and intersubtype variants may act as poor immunogens, presumably because the additional carbohydrate (CHO) occludes or limits exposure to the NtAb determinants on the envelope. Removal of key CHO residues results in immunogens that are more effective in eliciting intrasubtype NtAbs. This as been accomplished by removal of multiple N-linked carbohydrate sites in the SIV Env (Mori et al. 2001) and by removal of V2-associated carbohydrate in the HIV Env by deletion (Stamatatos and Cheng-Mayer 1998). These changes in Envelope make it more sensitive to neutralization in vitro (Stamatatos and Cheng-Mayer 1998; Stamatos et al. 1998); however, these immunogens are still only partially effective at generating intersubytpe NtAbs (Barnett et al. 2001). An alternative approach to broadening responses has been to immunized with Envelopes derived from different subtypes, resulting in some NtAbs in mice, but no evidence of significant cross-subtype immunity (Ljungberg et al. 2002). Recent efforts to expose more conserved determinants by fixing the conformation of Envelope have met with some success. Cross-linked Envelope and CD4 complexes have been shown to elicit broad intersubtype NtAbs in rhesus macaques (Fouts et al. 2002). Immunization of humans with their CD4 molecule may raise unwanted immune responses, due to the potential for autoimmune recognition of CD4-bearing cells. Therefore alternatives to presenting conserved conformational regions of Envelope are desirable.

### SUMMARY OF THE INVENTION

This invention provides a sequential vaccination protocol that directs the immune response away from the highly immunogenic variable regions toward the conserved determinants, thereby promoting generation of intra-subtype and inter-subtype primary HIV neutralizing antibodies (NtAbs). This result mimics the pattern of variants that arise in vivo but shortens the time needed to achieve the clinically beneficial result.

The subject vaccination protocol features immunization with a first HIV envelope immunogen having a minimum number of N- and O-linked glycosylation sites, followed by one or more booster immunizations with second HIV envelope immunogens having more glycosylation sites than the first immunogen.

The first immunogen preferably has from about 24 to 26 glycosylation sites in gp160, or 20 in gp120 and 4 in gp41. The following glycosylation sites are presently considered desirable: in gp120, positions 88, 130, 136, 156, 160, 197, 241, 262, 276, 296, 301, 332, 339, 355 or 356, 386, 392, 396 or 397, 405 or 406, 448, 463; and in gp41, positions 611, 616, 625, 637. This first immunogen may be derived from a natural clone from a primary HIV infection, or an engineered Envelope. In this manner the first immunogen (or second) can include gp160, gp140, or gp120, or likewise engineered constructs.

The second immunogen has at least one and preferably three or more additional glycosylation sites than the first immunogen. These additional sites may be located within hypervariable regions V1, V2, V3, V4, or V5 or constant region C2 or possibly C3. This second HIV Env immunogen may be derived from a natural clone from a late stage patient with intra- and inter-subtype primary HIV NtAbs or an engineered envelope.

More than one booster immunization may be employed. Preferably the subsequent immunogens will each bear increasing numbers of glycosylation sites, so that, for example, a third immunogen may bear 26-27 sites and the fourth may bear 28 sites or more. In general, the first immunogen should bear the least number of N- or O-linked glycosylation sites, and the last immunogen should bear the most. In addition to adding carbohydrate, it may be beneficial to maintain the same number of sites as found in the first immunogen, for example, V1, V2, and V4 but to shift the positions of these sites.

### BRIEF DESCRIPTION OF THE DRAWINGS

FIG. 1 shows the hypervariable regions and potential glycosylation sites on the HIV envelope, using the sequence of HIV-HXB2 (after Leonard et al. 1990). All numbering follows the conventional guidelines suggested by the Los Alamos National Laboratory group in 1998 entitled “Numbering Positions Relative to HXB2CG.”

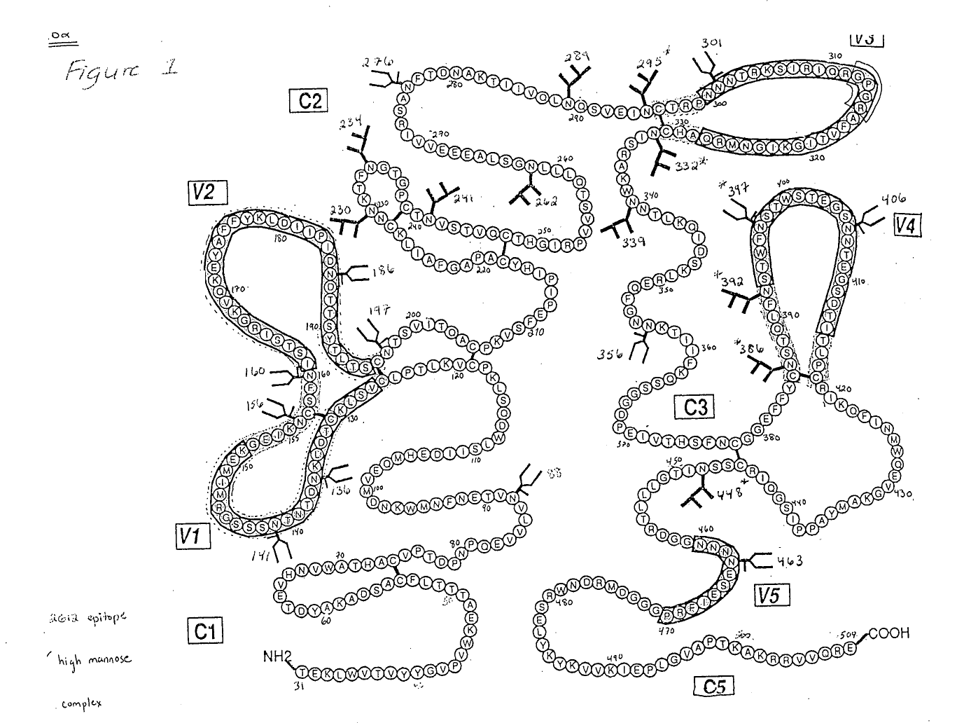

FIG. 2 shows the amino acid sequence of HIV-HXB2 with the positions of N-linked glycosylation sites found in Clade B (subtype B) NSI isolates. In this graphic representation, the darkest carbohydrate residues are those found in NSI (top). The positions of N-linked sites in the SI isolates are dark, for conserved with NSI, dotted, for those lost in SI, or lightly shaded, for those added in SI.

FIG. 3 shows the amino acid sequence of HIV-HXB2 with the positions of r N-linked glycosylation sites found in SHIV-SF162P4 Envelopes from Early (days 35-56), Middle (days 84-117), and Late (day 215) time points. The positions of N-linked sites in the early isolates are dark, for conserved with early, dotted, for those lost between early and middle or between middle and late, or lightly shaded, for those added from early to middle and from middle to late.

FIG. 4 shows the translated amino acid sequences of HIV Envelope from macaque A141 infected with SHIV-SF162P4 at specific days after infection in Variable regions 1 and 2 (V1/V2). Sequences are compared with the published HIV-SF162 env sequence. Sequence alignments are standard and show a “.” where there is identity and a new letter for any changes. Boxed “N” residues indicate those that are part of the canonical N-X-(S,T) N-linked glycosylation site. Sequences are for day 35 (8 clones); day 56 (10 clones); day 84 (9 clones); day 117 (14 clones); day 215 (8 clones).

FIG. 5 shows the translated amino acid sequence of HIV Envelope from macaque A141 as in FIG. 4. This alignment is of the 4^{th} Variable region (V4).

### DETAILED DESCRIPTION OF THE PREFERRED EMBODIMENT

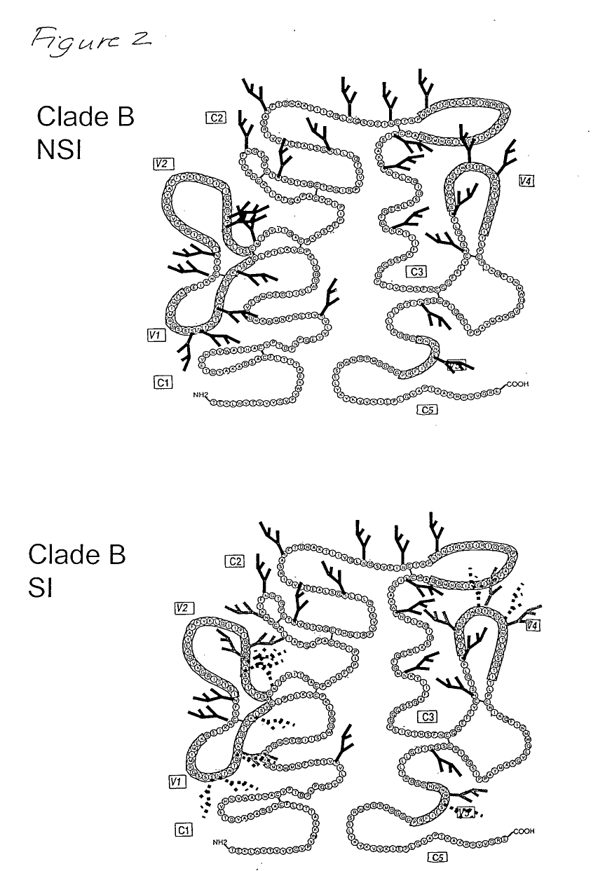

Our laboratory set out to determine whether changes in carbohydrate are important for altering the immunogenicity of Env. Relatively little data comparing early and late HIV Env sequences and changes over time from the same patients are available. We have analyzed the primary non syncytium-inducing (NSI) and late stage syncytium-inducing (SI) full length Env sequences from HIV-1 infected patients from the Los Alamos database (http://hiv-web.lanl.gov/content/hiv-db/ALIGN_CURRENT/ALIGN-INDEX.html). These sequences are not from the same patients, but represent average changes in sequences that are common to most NSI and SI viral Envelopes sequenced to date. Envelopes from primary HIV infections (NSI sequences) typically have less than about 24 N-linked glycosylation sites. The envelopes of late stage patients or SI virus sequences typically contain about 25 or more glycosylation sites. We note that changes result from both loss and gain of glycosylation sites, resulting in repositioning of carbohydrate in key regions. The glycosylation sites found in NSI and SI isolates are summarized in the table below (Table 1). Sequences were numbered according to the HXBc2 sequence, and occurrence of N-X-(S,T) at specific sites was scored for each sequence. Numbers listed indicate scores near 1. A summary of the primary data is provided in graphic form in FIG. 2. In this case, no information is available about neutralization sensitivity or the presence of neutralizing antibodies in the sera of the infected patients.

TABLE 1
Amino acid residues in constant (C) and variable (V) regions of Envlope
that are potential N-linked or O-linked glycosylation sites. Comparison of 25 NSI
and 16 SI HIV B subtype isolates from the Los Alamos Database. Positions noted are
those that are present in all or nearly all sequences compared. Numbers in
parentheses are present less than 100% time; underlined numbers are those that
differ between NSI and SI.

| HIV | Region of glycosylation site | | | | | | | | | |
|---|---|---|---|---|---|---|---|---|---|---|
| Type | C1 | V1 | V2 | C2 | V3 | C3 | V4 | C4 | V5 | 41 |

| | | | | | | | | | | |
|---|---|---|---|---|---|---|---|---|---|---|
| NSI | 88 | 136 | 160 | 197 | 301 | 332 | 386 | 448 | (461) | 611 |
| 24 | 130 | 141 | (188) | 234 | | 339 | 392 | | 463-4 | 616 |
| sites | | 156 | | 241 | | 355/6 | 396/7 | | | 625 |
| | | | | 262 | | (362) | 405/6 | | | 637 |
| | | | | 276 | | | | | | |
| | | | | 289 | | | | | | |
| | | | | 295 | | | | | | |
| SI | 88 | 136 | 160 | 197 | 301 | 332 | 386 | 448 | 463 | 611 |
| 25 | 130 | 141 | (186) | 230 | | 339 | 392 | | | 616 |
| sites | | 156 | | 234 | | 356 | (396/7) | | | 625 |
| | | | | 241 | | (362) | (402) | | | 637 |
| | | | | 262 | | | (406) | | | 816 |
| | | | | 276 | | | | | | |
| | | | | 289 | | | | | | |
| | | | | 295 | | | | | | |

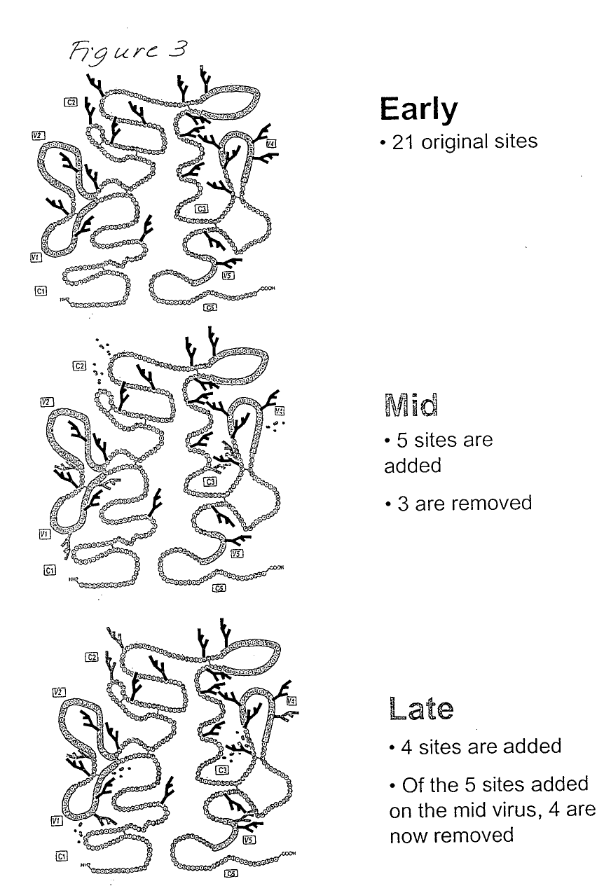

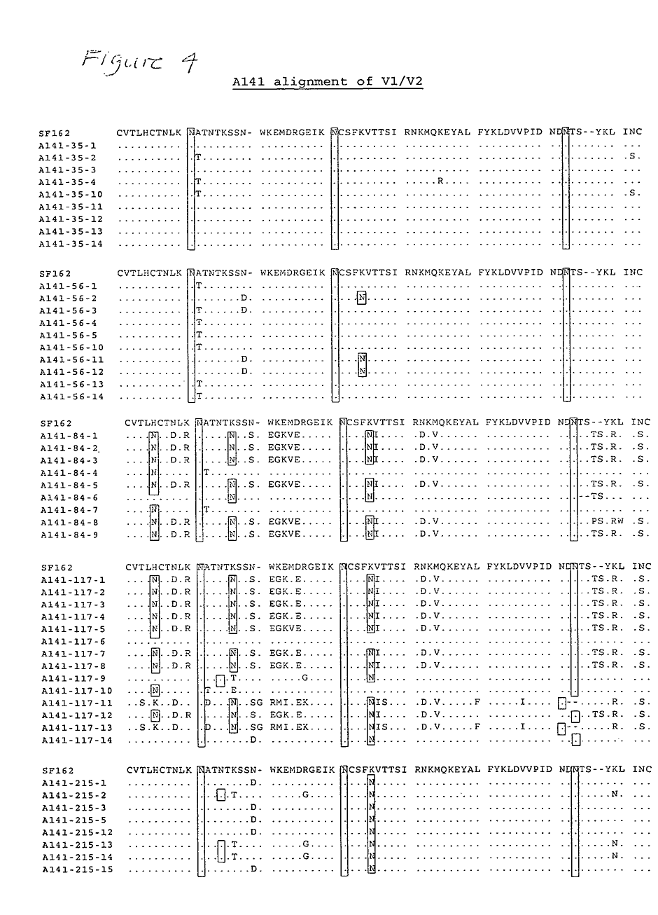

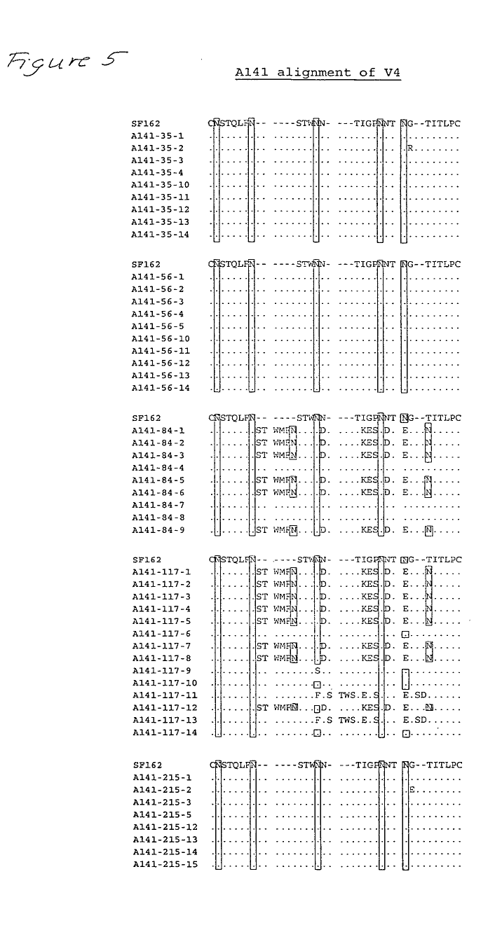

The chimeric virus SHIV bears the HIV env gene in the backbone of the SIV genome. SHIV infection of macaques has produced a number of SHIV isolates that cause pathogenesis in the macaque frame of 1 year or less. These macaques are subject to sequence diversification and develop NtAbs against viral variants, just as HIV-infected humans do. Comparison of the data from SHIV-infected macaques and HIV-infected humans shows remarkable conservation of glycosylation sites, and it also allows a comparison of sites that are altered over time in both types of infection. We have used the polymerase chain reaction (PCR) and HIV-1 env-specific primers to amplify the envelope gene gp160 or gp120 from DNA obtained from peripheral blood mononuclear cells of infected macaques. From these sequences, we inferred the amino acid sequence and analyzed these sequences for changes, and particularly for changes in N-linked glycosylation sites. The glycosylation site data summarized in Table 2 are from the first year samples from a SHIV-infected macaque with moderate disease progression and evidence of significant increases in magnitude and breadth of the NtAb response between day 56 and day 84. Changes to the glycosylation pattern are underlined in the day 84-117 data, showing the acquisition of 5 sites during quasispecies differentiation. Additional data are shown in FIGS. 3-5. We propose that the changes observed in glycosylation pattern sites are contributing to the broadening of the neutralizing antibody response in this animal.

TABLE 2
Amino acid residues in constant (C) and variable (V) regions of SHIV Envlope
that are potential N-linked or O-linked glycosylation sites. Summary of comparison of
multiple Envelope sequences from macaque A141, infected with SHIV-SF162P4, at
days 35 and 56 (early); at days 84 and 117 (middle). Positions noted are those that
are present in all or nearly all sequences compared. Numbers in parentheses are
present less than 100% time; underlined numbers are those are present at the later
time point and not present at the earlier time point.

| SHIV | Region of glycosylation site | | | | | | | | | |
|---|---|---|---|---|---|---|---|---|---|---|
| Days | C1 | V1 | V2 | C2 | V3 | C3 | V4 | C4 | V5 | 41 |
| 35-56 | 88 | 136 | | 197 | 301 | 332 | 386 | 448 | 463 | 611 |
| | | | 188 | 234 | | 339 | 392 | | | 616 |
| | | 156 | | 241 | | 355 | | | | 625 |
| | | | | 262 | | | 396 | | | 637 |
| | | | | 276 | | | 406 | | | |
| | | | | 295 | | | 409 | | | |
| 84-117 | 88 | 136 | 160 | 197 | 301 | 332 | 386 | 448 | 463 | 611 |
| | 130 | 141 | 188 | 234 | | 339 | 392 | | | 616 |
| | | 156 | | 241 | | 355 | 393 | | | 625 |
| | | | | 262 | | 362 | 396 | | | 637 |
| | | | | 276 | | | 406 | | | |
| | | | | 289 | | | 409 | | | |
| | | | | 295 | | | | | | |

### PRODUCTION OF ENV IMMUNOGENS (GENES AND PROTEINS)

Envelope genes can be obtained from patient virus samples by pelleting virus from plasma and extracting RNA, reverse transcribing the RNA into DNA, and specifically amplifying the Envelop DNA using DNA primers and the polymerase chain reaction (PCR) and Taq polymerase. Primers that amplify Envelope are situated outside the Envelope coding region and prime the entire length of the gene. Primers that amplify Envelope from many different HIV sequences are designed to bind to conserved regions of the viral genome outside the envelope coding region. In many cases, it may be necessary to use two rounds of PCR to obtain the Env sequence. In this case, the second set of primers will be situated inside the outer primers to make a “nested set” of sequences, where the first set will amplify a slightly larger product and the second nested set a smaller product.

Selection or Engineering of the 1^{ST} AND 2^{ND} Immunogens and Testing Criteria

By analysis of published and cloned sequences, the minimum number of N-linked glycosylation sites that are present on early infecting isolates has been deduced. These are usually but not exclusively located at positions note below (numbering system follows the convention of numbering from the HXB2 cloned sequence). The exact locations of these sites may vary slightly from one HIV isolate to another: SU gp120: 88, 130, 136, 156, 160, 197, 241, 262, 276, 296, 301, 332, 339, 355 or 356, 386, 392, 396 or 397, 405 or 406, 448, 463; n=20

TM gp41: 611, 616, 625, 637; n=4

If a cloned envelope gene encodes approximately this number of sites, it may be used as an immunogen directly. If the gene encodes more N-linked sites than approximately 24, then the first immunogen can be derived from the cloned gene by alteration to create appropriate mutations. Additional sites are removed by, for example, site-directed mutagenesis to change Asn residues to Gln residues. The resulting sequence should retain N-X-(S,T) sites at the key positions listed above, or at sites adjacent to these sites.

Second immunogen(s) can then be engineered by the addition of at least one and preferably 3 or more additional N-linked site at positions recommended below, if they are not already present in the first immunogen sequence:
C1:130, to provide a preferred total of 2 sites
V1:141, to provide a preferred total of 3 sites
V2:160 or 186-188, to provide a preferred total of 2 sites
C2:to provide a preferred total of up to 8 sites at positions noted
V3:1 site preferred at position 301
C3:362, to provide a preferred total of 4 sites or up to 5 sites
V4:393, 402, or 409, to provide a preferred total of 5 sites at positions noted
C4:1 site preferred at position 448 V5:2 sites preferred at 461 and 463
Gp41:to provide a preferred total of up to 5 sites at positions noted

In some cases it may be necessary to provide more than two different immunogens. Preferably the subsequent immunogens will each bear increasing numbers of glycosylation sites selected from these regions listed above, so that the third variant will bear 26-27 sites and the fourth immunogen will bear 28 sites or more. In general, the first immunogen should bear the least number of N- or O-linked glycosylation sites, and the last immunogen should bear the most. In some cases it may be beneficial to maintain the number of sites in V1, V2, and V4 and to shift the positions of these sites.

### USEFUL VACCINATION PROTOCOLS

Useful vaccination protocols may include recombinant vaccines that express the Env protein. Examples of this include recombinant viruses such as poxviruses (vaccinia virus, modified Vaccinia Ankara, and canarypox vectors); recombinant adenoviruses that are replication competent or replication incompetent; recombinant adenovirus-associated virus; Venezuelan Equine Encephalitis viruses; or Vesicualr Stomatatis Viruses, for example. Additional vaccines may include DNA expression vectors typically based on the human cytomegalovirus Immediate Early-I promoter and competent to direct Env expression in mammalian cells (“DNA vaccines”) that may be delivered by a variety of routes (intramuscular, intradermal, transdermal, oral, intravaginal, intrarectal, intranasal, etc.). Further, vaccines may consist of recombinant proteins that are delivered in adjuvants or in microspherical biodegradable particles to various sites using intramuscular or intradermal or oral immunization. These vaccines may be used alone or in combination with one another. Vaccination with Env may be accompanied by vaccination with other HIV proteins or other viral proteins at the same time using similar or identical vectors or systems to achieve responses to Env and the other proteins at the same time.

### TIME PERIOD FOR SEQUENTIAL ADMINISTRATION

Sequential delivery of vaccines for humans usually follows the time course of vaccination at week 0, week 4 (1 month) and week 24-26 (6 months). This time period of sequential immunization is adequate and may be improved by additional immunizations at 10 months and 12 months, for example.

### LABORATORY MEASURES OF SUCCESS

Success of the vaccination protocol can be measured by showing that sera or plasma from immunized test animals have antiviral Neutralizing Antibodies (associated with IgA, secretory IgA, or IgG) effective in blocking viral infection in vitro. The levels of NtAbs should increase with each additional immunization. Targets of these NtAbs will include at least a subset of HIV-1 primary isolates from the same and different viral subtypes. Typically, at least a majority of a panel of primary viruses from at least two different subtypes will be neutralized to at least the 50% level by a 1:10 dilution of the serum that is obtained 2 weeks after the last immunization. The choice of neutralizing assay will not influence the determination of the overall effectiveness of the vaccine, although it is well known that different assays have differing levels of sensitivity and that different viruses have different levels of sensitivity to NtAbs. Sera with high activity against multiple subtype primary HIV would be predicted to neutralize most representative primary HIV from many subtypes, regardless of the assay or the choice of primary viruses.

While the invention has been described in conjunction with preferred embodiments, one of ordinary skill after reading the foregoing specification will be able to effect various changes, substitutions of equivalents, and alterations to the subject matter set forth herein. Hence, the invention can be practiced in ways other than those specifically described herein.

### Citations

1. Barnett, S. W., S. Lu, et al. (2001). “The ability of an oligomeric human immunodeficiency virus type 1 (HIV-1) envelope antigen to elicit neutralizing antibodies against primary HIV-1 isolates is improved following partial deletion of the second hypervariable region.” J Virol 75(12): 5526-40.
2. Binley, J. M., R. W. Sanders, et al. (2000). “A recombinant human immunodeficiency virus type 1 envelope glycoprotein complex stabilized by an intermolecular disulfide bond between the gp120 and gp41 subunits is an antigenic mimic of the trimeric virion-associated structure.” J Virol 74(2): 627-43.
3. Burns, D. P. and R. C. Desrosiers (1994). “Envelope sequence variation, neutralizing antibodies, and primate lentivirus persistence.” Curr Top Microbiol Immunol 188:185-219.
4. Cecilia, D., C. Kleeberger, et al. (1999). “A longitudinal study of neutralizing antibodies and disease progression in HIV-1-infected subjects.” J Infect Dis 179(6):1365-74.
5. Chackerian, B., L. M. Rudensey, et al. (1997). “Specific N-linked and O-linked glycosylation modifications in the envelope V1 domain of simian immunodeficiency virus variants that evolve in the host alter recognition by neutralizing antibodies.” J Virol 71(10): 7719-27.
6. Fouts, T., K. Godfrey, et al. (2002). “Crosslinked HIV-1 envelope-CD4 receptor complexes elicit broadly cross-reactive neutralizing antibodies in rhesus macaques.” Proc Natl Acad Sci U S A 99(18): 11842-7.
7. Garrity, R. R., G. Rimmelzwaan, et al. (1997). “Refocusing neutralizing antibody response by targeted dampening of an immunodominant epitope.” J Immunol 159(1): 279-89.
8. Haigwood, N. L., P. L. Nara, et al. (1992). “Native but not denatured recombinant human immunodeficiency virus type 1 gp120 generates broad-spectrum neutralizing antibodies in baboons.” J Virol 66(1): 172-82.
9. Haigwood, N. L., J. R. Shuster, et al. (1990). “Importance of hypervariable regions of HIV-1 gp120 in the generation of virus neutralizing antibodies.” AIDS Res Hum Retroviruses 6(7): 855-69.
10. Hirsch, V. M., G. Dapolito, et al. (1998). “Viral genetic evolution in macaques infected with molecularly cloned simian immunodeficiency virus correlates with the extent of persistent viremia.” J Virol 72(8): 6482-9.
11. Kimata, J. T., L. Kuller, et al. (1999). “Emerging cytopathic and antigenic simian immunodeficiency virus variants influence AIDS progression.” Nat Med 5(5): 535-41.
12. Kimura, T., K. Yoshimura, et al. (2002). “Reconstitution of spontaneous neutralizing antibody response against autologous human immunodeficiency virus during highly active antiretroviral therapy.” J Infect Dis 185(1):53-60.
13. Leonard C K, Spellman M W, Riddle L, Harris R J, Thomas J N, Gregory T J (1990). “Assignment of intrachain disulfide bonds and characterization of potential glycosylation sites of the type 1 recombinant human immunodeficiency virus envelope glycoprotein (gp120) expressed in Chinese hamster ovary cells.” J Biol Chem 265 (18): 10373-82.
14. Ljungberg, K., E. Rollman, et al. (2002). “Enhanced Immune Responses After DNA Vaccination with Combined Envelope Genes from Different HIV-1 Subtypes.” Virology 302(1): 44-57.
15. Los Alamos National Laboratory HIV Sequence Database, Theoretical Biology and Physics Group T-10, Mail Stop K710, Los Alamos, N.M. 87545 http://hiv-web.lanl.gov/content/hiv-db/ALIGN_CURRENT/ALIGN-INDEX.html
16. Mascola, J. R., S. W. Snyder, et al. (1996). “Immunization with envelope subunit vaccine products elicits neutralizing antibodies against laboratory-adapted but not primary isolates of human immunodeficiency virus type 1. The National Institute of Allergy and Infectious Diseases AIDS Vaccine Evaluation Group.” J Infect Dis 173(2): 340-8.
17. Moog, C., H. J. A. Fleury, et al. (1997). “Autologous and heterologous neutralizing antibody responses following initial seroconversion in human immunodeficiency virus type 1-infected individuals.” Journal of Virology 71: 3734-3741.
18. Mori, K., Y. Yasutomi, et al. (2001). “Quintuple deglycosylation mutant of simian immunodeficiency virus SIVmac239 in rhesus macaques: robust primary replication, tightly contained chronic infection, and elicitation of potent immunity against the parental wild-type strain.” J Virol 75(9): 4023-8.
19. Stamatatos, L. and C. Cheng-Mayer (1998). “An envelope modification that renders a primary, neutralization-resistant clade B human immunodeficiency virus type 1 isolate highly susceptible to neutralization by sera from other clades.” J Virol 72(10):7840-5.
20. Stamatatos, L., M. Lim, et al. (2000). “Generation and structural analysis of soluble oligomeric gp140 envelope proteins derived from neutralization-resistant and neutralization-susceptible primary HIV type 1 isolates.” AIDS Res Hum Retroviruses 16(10): 981-94.
21. Stamatos, N. M., J. R. Mascola, et al. (1998). “Neutralizing antibodies from the sera of human immunodeficiency virus type 1-infected individuals bind to monomeric gp120 and oligomeric gp140.” J Virol 72(12):9656-67.
22. Steimer, K. S., C. J. Scandella, et al. (1991). “Neutralization of divergent HIV-1 isolates by conformation-dependent human antibodies to Gp120.” Science 254(5028):105-8.
23. Sullivan, N., Y. Sun, et al. (1998). “CD4-Induced conformational changes in the human immunodeficiency virus type 1 gp120 glycoprotein: consequences for virus entry and neutralization.” J Virol 72(6): 4694-703.
24. Sullivan, N., M. Thali, et al. (1993). “Effect of amino acid changes in the V1/N2 region of the human immunodeficiency virus type 1 gp 120 glycoprotein on subunit association, syncytium formation, and recognition by a neutralizing antibody.” J Virol 67(6): 3674-9.
25. Wang, F. X., T. Kimura, et al. (2002). “Emergence of autologous neutralization-resistant variants from preexisting human immunodeficiency virus (HIV) quasi species during virus rebound in HIV type 1-infected patients undergoing highly active antiretroviral therapy.” J Infect Dis 185(5): 608-17.
26. Wyatt, R., J. Moore, et al. (1995). “Involvement of the V1/V2 variable loop structure in the exposure of human immunodeficiency virus type 1 gp120 epitopes induced by receptor binding.” J Virol 69(9): 5723-33.

1. A vaccination protocol comprising immunizing a mammalian host with a first HIV envelope immunogen having a minimum number of N- and O-linked glycosylation sites, followed by one or more booster immunizations with at least one second HIV envelope immunogen having more glycosylation sites than the first immunogen.
